(19) United States
(12) Patent Application Publication
(10) Pub. No.: US 2025/0107694 A1
(43) Pub. Date: Apr. 3, 2025

(54) ENDOSCOPE

(71) Applicant: FUJIFILM Corporation, Tokyo (JP)

(72) Inventors: Takashi HARADA, Kanagawa (JP); Shozo IYAMA, Kanagawa (JP)

(73) Assignee: FUJIFILM Corporation, Tokyo (JP)

(21) Appl. No.: 18/900,572

(22) Filed: Sep. 27, 2024

(30) Foreign Application Priority Data
Sep. 29, 2023 (JP) 2023-169944

(51) Int. Cl. A61B 1/00; A61B 1/12

(52) U.S. Cl. CPC A61B 1/00133; A61B 1/0008; A61B 1/00114; A61B 1/12

(57) ABSTRACT

Provided is an endoscope capable of improving cleanability of a distal end portion of an endoscope insertion part. An endoscope includes: a distal end portion including an elevator housing chamber and a lever housing chamber; an elevator housed in the elevator housing chamber; a lever housed in the lever housing chamber; an operation wire connected to the lever; a first partition wall that is provided at the distal end portion and that divides the elevator housing chamber and the lever housing chamber; a rotary shaft that couples the elevator and the lever to each other, the rotary shaft being inserted through a through-hole provided in the first partition wall; and a sealing member that is mounted on the rotary shaft and that seals the through-hole in a case in which the rotary shaft is inserted through the through-hole, the sealing member being attachable and detachable from a lever housing chamber side.

### CROSS-REFERENCE TO RELATED APPLICATION

The present application claims priority under 35 U.S.C § 119 (a) to Japanese Patent Application No. 2023-169944 filed on Sep. 29, 2023, which is hereby expressly incorporated by reference, in its entirety, into the present application.

### BACKGROUND OF THE INVENTION

### 1. Field of the Invention

The present invention relates to an endoscope, and particularly, to an endoscope comprising an elevator at a distal end portion of an endoscope insertion part.

### 2. Description of the Related Art

In an endoscope, various treatment tools are inserted from a treatment tool inlet port provided at an operation part and are led out from a treatment tool outlet port that is open at a distal end portion of an insertion part, to be used for treatment. For example, in a duodenoscope, treatment tools such as a guide wire or a shaping tube are used. Such a treatment tool needs to change a lead-out direction thereof at the distal end portion of the insertion part in order to treat a desired position in a subject. Therefore, a treatment tool elevation mechanism (elevator) is provided at the distal end portion.

As the treatment tool elevation mechanism, a mechanism (open type) is known in which a wire is attached to the elevator, and the wire is extended to the operation part of the endoscope, and the elevator is swung around a rotary shaft by pushing and pulling the wire with an operation lever provided at the operation part, to change its position between an elevated position and a reclined position.

In addition, a mechanism (closed type) is also known in which the rotary shaft of the elevator is coupled to a housed lever via a partition wall, a wire is attached to the lever, and the elevator is swung around the rotary shaft by pushing and pulling the wire with the operation lever of the operation part, to change its position between the elevated position and the reclined position.

Since the distal end portion provided with such a treatment tool elevation mechanism has a complicated shape and structure, improved cleanability by a cleaning liquid is required.

CN209951207Y discloses an endoscope comprising a closed type treatment tool elevation mechanism in which a cleaning channel is formed between a through-hole provided in a partition wall and a rotary shaft inserted through the through-hole. According to the endoscope of CN209951207Y, the cleaning liquid flows into the cleaning channel and reaches a side surface of an elevator, thereby cleaning the rotary shaft, the through-hole, and the side surface of the elevator.

### SUMMARY OF THE INVENTION

In the endoscope of CN209951207Y, an elevator housing part and a lever housing part provided at a distal end portion of an endoscope insertion part are divided by the partition wall, and the rotary shaft is inserted through the through-hole provided in the partition wall to couple the elevator and the lever to each other. A cleaning channel for allowing the cleaning liquid to flow is formed between the through-hole and the rotary shaft.

Therefore, in the endoscope of CN209951207Y, during use of the endoscope, a liquid such as a body fluid that has entered the elevator housing part is likely to enter the lever housing part via the above-described cleaning channel. Therefore, the lever, the wire, and the like housed in the lever housing part may be contaminated by the above-described liquid. In such a case, the lever, the wire, and the like should be disassembled from the distal end portion and cleaned, making cleaning work complicated and also posing a problem in achieving complete cleaning.

The present invention has been made in view of such circumstances, and an object of the present invention is to provide an endoscope capable of improving cleanability of a distal end portion of an endoscope insertion part.

According to a first aspect, there is provided an endoscope comprising: a distal end portion including an elevator housing part and a lever housing part; an elevator housed in the elevator housing part; a lever housed in the lever housing part; a wire connected to the lever; a partition wall that is provided at the distal end portion and that divides the elevator housing part and the lever housing part; a rotary shaft that couples the elevator and the lever to each other, the rotary shaft being inserted through a first through-hole provided in the partition wall; and a sealing member that is mounted on the rotary shaft and that seals the first through-hole in a case in which the rotary shaft is inserted through the first through-hole, the sealing member being attachable and detachable from a lever housing part side.

According to a second aspect, in the endoscope described in the first aspect, the lever includes a second through-hole through which the rotary shaft is inserted, the rotary shaft is formed with a side part in which a peripheral surface parallel to an axial direction of the rotary shaft forms a planar shape, and an arc part in which the peripheral surface forms a curved shape, the arc part is in sliding contact with an inner surface of each of the first through-hole and the second through-hole, a void portion is provided between the side part and the inner surface of each of the first through-hole and the second through-hole, and the sealing member is fitted into the void portion.

According to a third aspect, in the endoscope described in the second aspect, the sealing member includes a base portion that is fitted to the lever to be non-rotatable relative to each other, and a protruding portion protruding from the base portion, and the protruding portion is fitted into the void portion.

According to a fourth aspect, in the endoscope described in the third aspect, the base portion includes a fitting hole to which the rotary shaft is fitted to be non-rotatable relative to each other.

According to a fifth aspect, in the endoscope described in the first aspect, a first rotary shaft that is provided on the elevator and that constitutes the rotary shaft, and a second rotary shaft that is provided on the lever and that constitutes the rotary shaft are further provided, and a fitting portion is provided on one of the first rotary shaft or the second rotary shaft, and a fitted portion that is fitted to the fitting portion is provided on the other rotary shaft different from the one rotary shaft.

According to a sixth aspect, in the endoscope described in the fifth aspect, the sealing member is attachable to and detachable from at least the second rotary shaft.

According to a seventh aspect, in the endoscope described in the fifth or sixth aspect, a knob portion is provided to protrude at an end part of the sealing member on the lever housing part side.

According to an eighth aspect, in the endoscope described in the fifth aspect, the sealing member is fixed to the second rotary shaft.

According to a ninth aspect, in the endoscope described in the fifth aspect, the sealing member and the second rotary shaft are an integrally formed article.

According to a tenth aspect, in the endoscope described in any one of the fifth to seventh aspects, the sealing member has a tubular shape through which the first rotary shaft and the second rotary shaft are inserted, and the sealing member includes a first flange portion that is provided on a first rotary shaft side in an axial direction of the sealing member and that protrudes in a radial direction orthogonal to the axial direction, and a second flange portion that is provided on a second rotary shaft side in the axial direction and that protrudes in the radial direction.

According to an eleventh aspect, in the endoscope described in the tenth aspect, a knob portion is provided to protrude at an end part of the second flange portion on the lever housing part side.

According to a twelfth aspect, in the endoscope described in the tenth or eleventh aspect, the first flange portion is disposed in the first through-hole, and the second flange portion is disposed in the lever housing part.

According to a thirteenth aspect, in the endoscope described in any one of the first to twelfth aspects, the elevator includes a third through-hole into which the rotary shaft is inserted, the rotary shaft is formed with a side part in which a peripheral surface parallel to an axial direction of the rotary shaft forms a planar shape, and an arc part in which the peripheral surface forms a curved shape, and the third through-hole includes an inner peripheral portion formed with a hole-side side part that faces and is in contact with the side part and a hole-side arc part that faces the arc part with a void portion therebetween.

According to the present invention, it is possible to improve cleanability of the distal end portion of the endoscope insertion part.

### BRIEF DESCRIPTION OF THE DRAWINGS

FIG. 1 is a perspective view of an assembly showing a configuration of a distal end portion of an endoscope insertion part.

FIG. 2 is a perspective view showing a structure of a distal end portion body.

FIG. 3 is a schematic front view of the distal end portion body.

FIG. 4 is an assembly view of the distal end portion body.

FIGS. 5A and 5B are explanatory views of a movement of an elevator moved by an operation of a lever.

FIG. 6 is a perspective view of an appearance of the elevator.

FIG. 7 is a perspective view of an assembly showing a coupling structure between the elevator and the lever.

FIG. 8 is a cross-sectional view of the coupling structure shown in FIG. 7.

FIG. 9 is a cross-sectional view of a rotary shaft and a partition wall-side through-hole.

FIG. 10 is an explanatory view of a void portion defined by the through-hole and the rotary shaft.

FIG. 11 is a perspective view of a sealing member mounted on the rotary shaft.

FIG. 12 is a front view of the sealing member.

FIG. 13 is a cross-sectional view of three protruding portions respectively fitted into three void portions.

FIG. 14 is a cross-sectional view of the rotary shaft and an elevator-side through-hole.

FIG. 15 is a cross-sectional view showing a first modification example of the coupling structure between the elevator and the lever.

FIG. 16 is a perspective view of a sealing member employed in the coupling structure of FIG. 15.

FIG. 17 is a front view of a base portion of the sealing member shown in FIG. 16.

FIG. 18 is a side view of the lever shown in FIG. 15.

FIG. 19 is a cross-sectional view showing a second modification example of the coupling structure between the elevator and the lever.

FIG. 20 is a perspective view of a sealing member employed in the coupling structure of FIG. 19.

FIG. 21 is a cross-sectional view showing a main part structure of a distal end portion according to a second embodiment.

FIG. 22 is a perspective view of an assembly of the coupling structure shown in FIG. 21.

FIG. 23 is a cross-sectional view of the sealing member fixed to a lever-side rotary shaft with an adhesive.

FIG. 24 is a cross-sectional view of the sealing member and the lever-side rotary shaft that are configured as an integrally formed article.

### DESCRIPTION OF THE PREFERRED EMBODIMENTS

Hereinafter, an endoscope according to embodiments of the present invention will be described.

In the embodiments, a side-viewing endoscope used as a duodenoscope is exemplified. The endoscope comprises an operation part that is operated by an operator and an endoscope insertion part (hereinafter, referred to as an “insertion part”) that is connected to the operation part and that is inserted into a subject.

The insertion part is inserted into the subject through an oral cavity and is further inserted into a duodenum from an esophagus through a stomach. As a result, a predetermined examination or treatment, such as medical therapy, using a treatment tool inserted into the insertion part is performed on the duodenum.

Examples of the treatment tool include biopsy forceps whose distal end portion includes a cup that allows collection of a biological tissue, a knife for endoscopic sphincterotomy (EST), or a contrast tube.

### First Embodiment

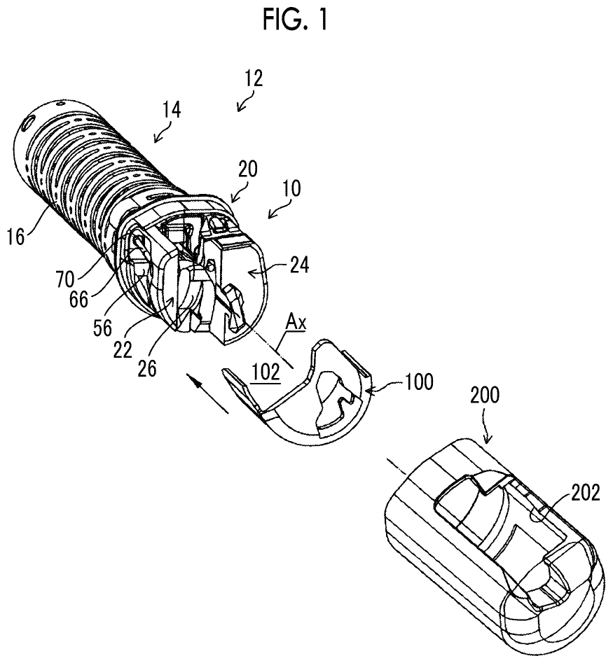

FIG. 1 is a perspective view of an assembly showing a configuration of a distal end portion 10 according to a first embodiment. The distal end portion 10 is provided on a distal end side of an insertion part 12. The distal end portion 10 is an example of a distal end portion of the embodiment of the present invention.

The insertion part 12 has a major axis Ax extending from a proximal end side connected to the operation part toward the distal end side and comprises a soft portion, a bending portion 14, and the distal end portion 10 in this order from the proximal end side toward the distal end side.

The bending portion 14 includes a cylindrical member 16 that is configured to bend by providing notch portions in a circumferential direction, and has a configuration in which an outer periphery of the cylindrical member 16 is covered with a mesh-like body woven with a metal wire and is further covered with a rubber outer sheath. In addition, a plurality of wires extend from an angle knob of the operation part to the bending portion 14, and distal end portions of these wires are fixed to the cylindrical member 16 constituting the bending portion 14. As a result, the bending portion 14 bends up, down, left, and right in response to an operation of the angle knob.

The distal end portion 10 includes a distal end portion body 20, a distal end cover 100 that is attachable to and detachable from the distal end portion body 20, and a cap 200 that is mounted on an outer side of the distal end cover 100.

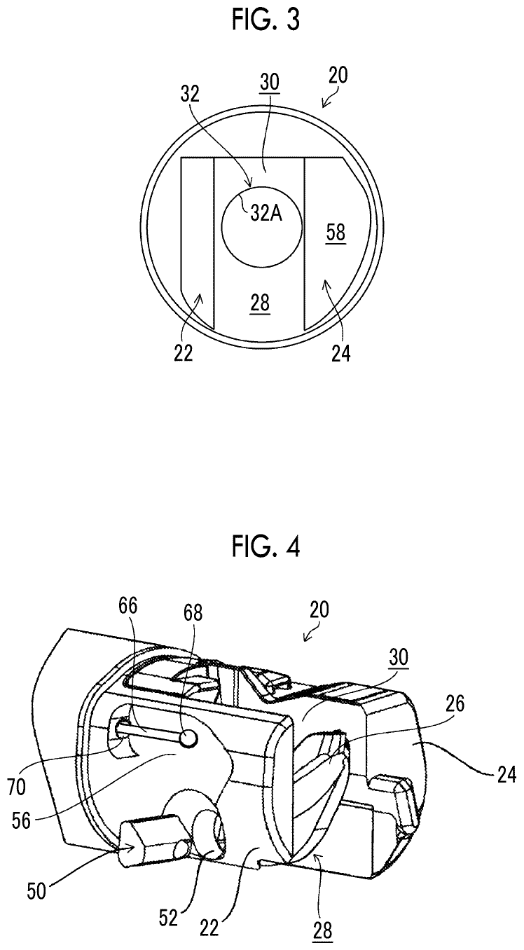

The distal end cover 100 includes an open part 102 through which an opening 30 on an upper surface side of an elevator housing chamber 28 (refer to FIG. 3), which will be described below, is open in a state in which the distal end cover 100 is mounted on the distal end portion body 20. The distal end cover 100 is made of a metal material having corrosion resistance.

In addition, the cap 200 includes an opening window 202 through which the above-described opening 30 is open in a state in which the cap 200 is mounted on the distal end portion body 20. The cap 200 includes an engaging portion that is provided on the proximal end side thereof and that is engaged with a groove formed in the distal end portion body 20, and is attachably and detachably mounted on the distal end portion body 20. The cap 200 consists of an elastic material or a flexible material, for example, silicone rubber or polyethylene.

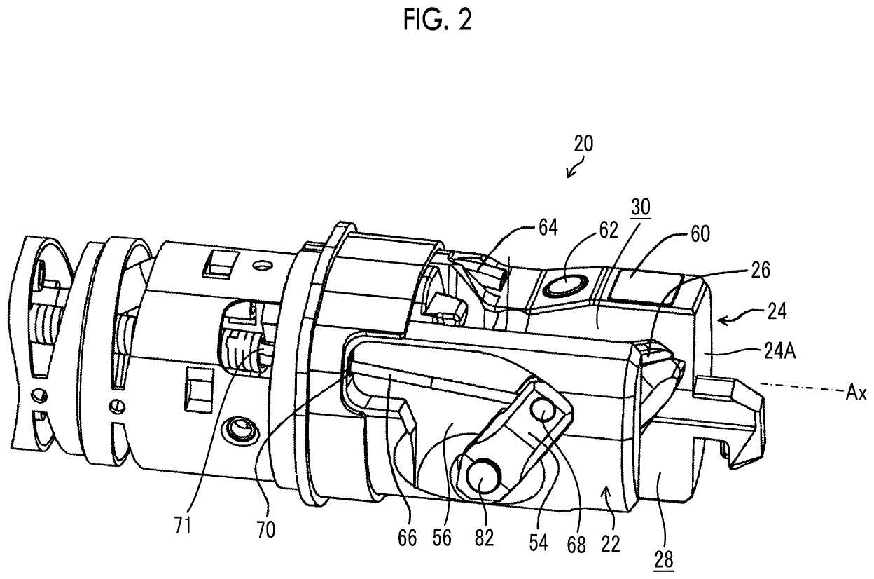

FIG. 2 is a perspective view of the distal end portion body 20. FIG. 3 is a schematic front view of the distal end portion body 20.

As shown in FIGS. 2 and 3, the distal end portion body 20 includes a pair of first and second partition walls 22 and 24 that are a part of the distal end portion body 20 and that face each other. The elevator housing chamber 28, which is a slit-shaped space for housing an elevator 26 (not shown in FIG. 3), is formed between the first partition wall 22 and the second partition wall 24. An opening of the elevator housing chamber 28 on the upper surface side in FIG. 2 is formed as the above-described opening 30. The distal end portion body 20 is made of a metal material having corrosion resistance. The elevator 26 and the elevator housing chamber 28 are examples of an elevator and an elevator housing part of the embodiment of the present invention.

In addition, a treatment tool insertion channel 32 communicates with the elevator housing chamber 28 of the distal end portion body 20. The treatment tool insertion channel 32 is inserted into the insertion part 12 and connected to a treatment tool inlet port of the operation part. As a result, the treatment tool introduced from the treatment tool inlet port is led out to the elevator housing chamber 28 through the treatment tool insertion channel 32.

The elevator 26 is provided on the distal end portion body 20 and is housed in the elevator housing chamber 28. The elevator 26 changes a direction of the treatment tool led out to the elevator housing chamber 28 from a treatment tool outlet port 32A, which is a distal end opening of the treatment tool insertion channel 32, causing the treatment tool to protrude through the opening 30. The elevator 26 is swingably attached to a through-hole 52 of the first partition wall 22 via a rotary shaft 50 (refer to FIG. 4), which will be described below, and can control a lead-out direction of the treatment tool in a case in which the treatment tool is led out from the treatment tool outlet port 32A.

A recessed lever housing chamber 56 that houses a lever 54 is formed on a surface of the first partition wall 22 on a side opposite to a facing surface side facing the elevator housing chamber 28. That is, the distal end portion 10 includes the elevator housing chamber 28 and the lever housing chamber 56. In addition, the first partition wall 22 that divides the elevator housing chamber 28 and the lever housing chamber 56 is provided at the distal end portion 10. The lever 54, the lever housing chamber 56, and the first partition wall 22 are examples of a lever, a lever housing part, and a partition wall of the embodiment of the present invention.

A housing chamber 58 that houses an optical system and the like is provided in the second partition wall 24. An irradiation window 60, an observation window 62, and an air and water supply nozzle 64 directed toward the observation window 62 are provided at an upper part of the housing chamber 58. The air and water supply nozzle 64 is connected to an air and water supply device via an air and water supply tube inserted into the insertion part 12. By operating an air and water supply button of the operation part, air or water is jetted from the air and water supply nozzle 64 toward the observation window 62, and the observation window 62 is cleaned.

In addition, an illumination unit and an imaging unit are housed in the second partition wall 24. The illumination unit comprises an illumination lens installed on an inner side of the irradiation window 60 and a light guide whose distal end is disposed to face the illumination lens. The light guide is inserted into the insertion part 12, and a proximal end portion thereof is connected to a light source device. As a result, irradiation light from the light source device is transmitted via the light guide and is emitted from the irradiation window 60.

The imaging unit comprises an imaging optical system disposed on an inner side of the observation window 62, and a complementary metal-oxide-semiconductor (CMOS) type imaging element or a charge-coupled device (CCD) type imaging element. The imaging element is connected to an image processing device via a signal cable inserted into the insertion part 12. An imaging signal of a subject image obtained by the imaging using the imaging unit is input to the image processing device via the signal cable, and the subject image is displayed on a monitor connected to the image processing device.

FIG. 4 is a perspective view of an assembly of the distal end portion body 20. In FIG. 4, the lever 54 is not shown, and the rotary shaft 50 to be connected to the lever 54 is shown.

As shown in FIG. 4, the through-hole 52 that penetrates the first partition wall 22 and that communicates with the elevator housing chamber 28 is provided in a bottom surface of the recessed lever housing chamber 56. The rotary shaft 50 that couples the elevator 26 and the lever 54 to each other is inserted through the through-hole 52. That is, the first partition wall 22 is provided with the through-hole 52 through which the rotary shaft 50 is inserted. Since the lever 54 in the lever housing chamber 56 swings about the rotary shaft 50, the lever housing chamber 56 is formed in a substantially fan shape centered on the rotary shaft 50. The rotary shaft 50 and the through-hole 52 are examples of a rotary shaft and a first through-hole of the embodiment of the present invention.

As will be described in a detailed configuration below, one end side of the lever 54 is coupled to the elevator 26 via the rotary shaft 50 and the other end side thereof is connected to an operation wire 66. The lever 54 swings about the rotary shaft 50 integrally with the elevator 26 with operation of the operation wire 66.

As shown in FIG. 2, the operation wire 66 includes a connection portion 68 that is provided on a distal end side of the operation wire 66 and that is connected to the lever 54 in the lever housing chamber 56. In addition, a proximal end side of the operation wire 66 is coupled to an elevator operating mechanism in the operation part through a wire insertion passage 71 from an outlet 70 that is open at a wall surface of the lever housing chamber 56. The operation wire 66 is an example of a wire of the embodiment of the present invention.

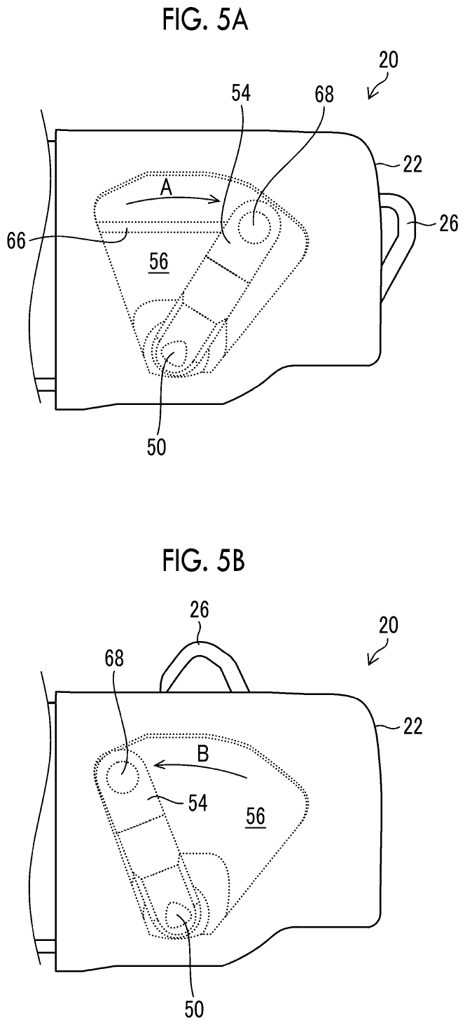

FIGS. 5A and 5B are explanatory views illustrating movements of the elevator 26 moved by an operation of the lever 54.

As shown in FIG. 5A, in a case in which the operation wire 66 is pushed by an operation of an operation lever, the lever 54 swings in an A direction about the rotary shaft 50, and the elevator 26 moves from an elevated position toward a reclined position. On the other hand, as shown in FIG. 5B, in a case in which the operation wire 66 is pulled by the operation of the operation lever, the lever 54 swings in a B direction (a direction opposite to the A direction) about the rotary shaft 50, and the elevator 26 moves from the reclined position to the elevated position. In this way, the operation wire 66 can rotationally move the rotary shaft 50 via the lever 54 through the operation of the operation lever. As a result, a position of the elevator 26 is changed between the elevated position and the reclined position.

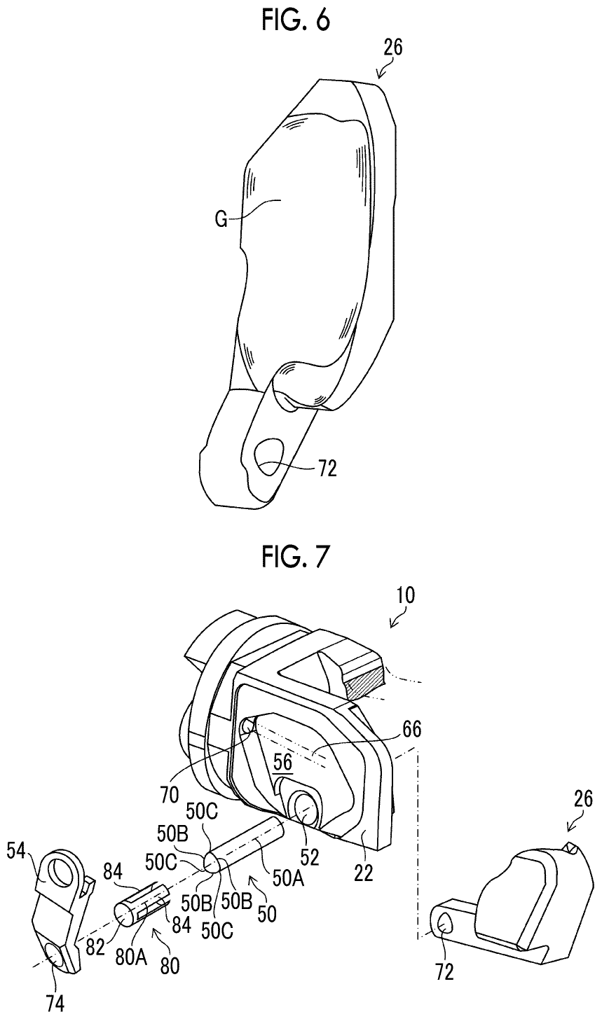

FIG. 6 is a perspective view of an appearance of the elevator 26 housed in the elevator housing chamber 28.

As shown in FIG. 6, the elevator 26 includes a circular arc-shaped guide surface G. The guide surface G faces the treatment tool outlet port 32A of the treatment tool insertion channel 32 in the elevator housing chamber 28 and guides, toward the opening 30, the treatment tool led out from the treatment tool outlet port 32A to the elevator housing chamber 28.

A proximal end portion of the elevator 26 is provided with a through-hole 72 into which the rotary shaft 50 is inserted. The rotary shaft 50 is inserted into the through-hole 72, whereby the elevator 26 is coupled to the lever 54 via the rotary shaft 50. The rotary shaft 50 and the through-hole 72 will be described below.

Here, the distal end portion 10 of the insertion part 12 is a member that is exposed to a liquid such as a body fluid during use of the endoscope. Therefore, the liquid may flow from the elevator housing chamber 28 to the through-hole 52 of the first partition wall 22 and may enter the lever housing chamber 56. In this case, since the rotary shaft 50, the through-hole 52, the lever 54, and the like may be contaminated by the liquid, these components should be cleaned. However, it is difficult to completely clean these components because these components are small and have complicated shapes.

In that respect, the distal end portion 10 of the first embodiment has the following configuration (coupling structure) in order to solve the above-described problem of the cleanability and to improve the cleanability of the distal end portion 10.

### Coupling Structure Between Elevator and Lever

FIG. 7 is a perspective view of an assembly showing a coupling structure between the elevator 26 and the lever 54. FIG. 8 is a cross-sectional view of the coupling structure shown in FIG. 7.

### Rotary Shaft

First, the rotary shaft 50 will be described. The rotary shaft 50 is formed with a side part 50B in which a peripheral surface parallel to a direction of an axis 50A of the rotary shaft 50 forms a planar shape, and an arc part 50C in which the peripheral surface forms a curved shape. Specifically, the rotary shaft 50 has a substantially triangular prism shape in which three side parts 50B and three arc parts 50C are alternately formed in a peripheral direction of the rotary shaft 50 and the side parts 50B and the arc parts 50C are extended along the direction of the axis 50A. The rotary shaft 50, the side part 50B, and the arc part 50C are examples of a rotary shaft, a side part, and an arc part of the embodiment of the present invention.

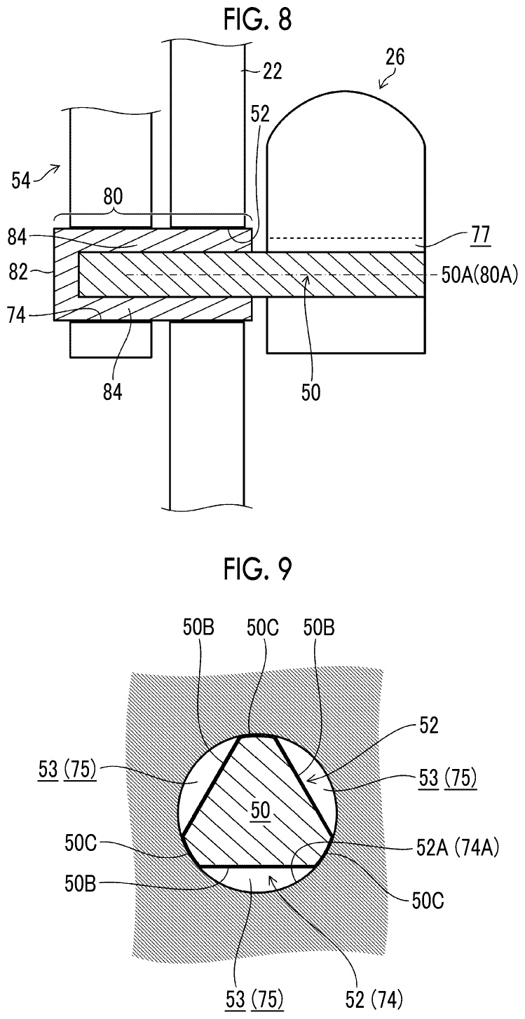

In a case in which such a rotary shaft 50 is inserted through the through-hole 52 in order to couple the elevator 26 and the lever 54 to each other, as shown in the cross-sectional view of the rotary shaft 50 and the through-hole 52 shown in FIG. 9, the arc part 50C of the rotary shaft 50 is in sliding contact with an inner surface 52A of the through-hole 52, which is a round hole, but the side part 50B of the rotary shaft 50 is not in contact with the inner surface 52A. Therefore, a void portion 53 is formed between the side part 50B and the inner surface 52A. In the present example, the void portion 53 is formed at three locations in the peripheral direction of the rotary shaft 50. The void portion 53 is an example of a void portion of the embodiment of the present invention.

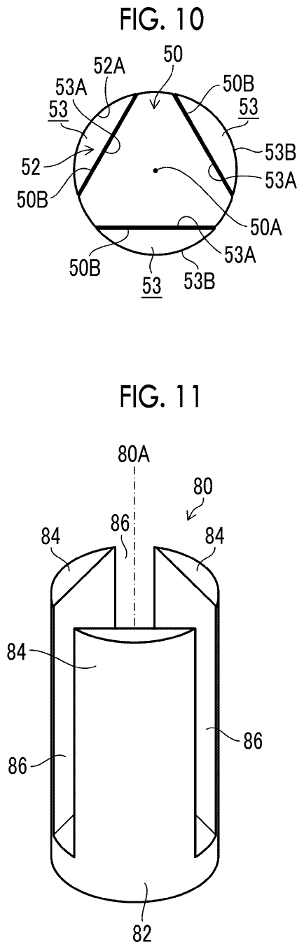

FIG. 10 is an explanatory view of the void portion 53 provided between the side part 50B of the rotary shaft 50 and the inner surface 52A of the through-hole 52.

As shown in FIG. 10, the void portion 53 is a space portion defined by the side part 50B and the inner surface 52A and is formed in a substantially semilunar shape having a void-side side part 53A along the side part 50B and a void-side arc part 53B along the inner surface 52A.

Since the void portion 53 formed in this way serves as a flow passage of the fluid from the elevator housing chamber 28 toward the lever housing chamber 56, it is necessary to close the void portion 53 and seal the through-hole 52 in order to improve the cleanability of the distal end portion 10. In that respect, the distal end portion 10 of the first embodiment comprises a sealing member 80 (refer to FIG. 11) for sealing the through-hole 52 in the coupling structure between the elevator 26 and the lever 54. Hereinafter, the sealing member 80 will be described.

### Sealing Member

FIG. 11 is an enlarged perspective view of the sealing member 80 mounted on the rotary shaft 50 shown in FIGS. 7 and 8.

As shown in FIG. 11, the sealing member 80 is configured in a substantially tubular shape having an axis 80A. The sealing member 80 comprises a base portion 82 and three protruding portions 84 protruding from the base portion 82. As will be described below, the three protruding portions 84 are configured to be fitted into the three void portions 53 shown in FIG. 10. The sealing member 80 is made of plastic such as polytetrafluoroethylene (PTFE), or rubber such as fluororubber and silicone rubber. The sealing member 80 is an example of a sealing member of the embodiment of the present invention.

Specifically, the base portion 82 of the sealing member 80 is configured in a disk shape. The three protruding portions 84 protrude parallel to each other from the base portion 82 along a direction of the axis 80A. Outer peripheral surfaces of the base portion 82 and each of the three protruding portions 84 are flush with each other without any steps, and the three protruding portions 84 are configured in a plate shape extending from the outer peripheral surface of the base portion 82 in the direction of the axis 80A. In addition, the three protruding portions 84 protrude at equal intervals in a peripheral direction around the axis 80A, with three slits 86 along the axis 80A therebetween.

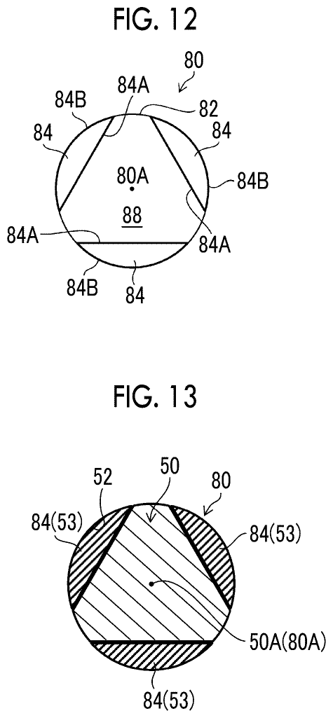

FIG. 12 is a front view of the sealing member 80 as seen from the direction of the axis 80A.

As shown in FIG. 12, the sealing member 80 includes a shaft housing space 88 surrounded by the three protruding portions 84. The rotary shaft 50 is housed in the shaft housing space 88. In this case, the rotary shaft 50 is housed in a state in which the axis 50A of the rotary shaft 50 is aligned with the axis 80A of the sealing member 80. As a result, the sealing member 80 is mounted on the rotary shaft 50.

The protruding portion 84 includes an inner peripheral portion formed with a protruding-side side part 84A that faces and is in contact with the side part 50B of the rotary shaft 50, and an outer peripheral portion formed with a protruding-side arc part 84B that faces and is in sliding contact with the inner surface 52A of the through-hole 52. As a result, the protruding portion 84 is formed in a substantially semilunar shape including the protruding-side side part 84A along the void-side side part 53A shown in FIG. 10 and the protruding-side arc part 84B along the void-side arc part 53B.

With such a shape of the protruding portion 84, the protruding portion 84 can be fitted into the void portion 53. Therefore, in a case in which the rotary shaft 50 is inserted through the through-hole 52, the three protruding portions 84 of the sealing member 80 mounted on the rotary shaft 50 are fitted into the three void portions 53, respectively. FIG. 13 is a cross-sectional view of the three protruding portions 84 respectively fitted into the three void portions 53.

Consequently, the three void portions 53 are closed by the three protruding portions 84, respectively, so that the through-hole 52 can be sealed. Accordingly, the sealing member 80 can block the flow of the fluid from the elevator housing chamber 28 toward the lever housing chamber 56.

Meanwhile, as shown in FIG. 8, the lever 54 is also provided with a through-hole 74. The sealing member 80 mounted on the rotary shaft 50 is also inserted through the through-hole 74. The through-hole 74 has a slightly smaller inner diameter than an inner diameter of the through-hole 52. A disposition relationship between the rotary shaft 50 and the through-hole 74 is similar to a disposition relationship between the rotary shaft 50 and the through-hole 52 shown in FIG. 9, although there is a difference in the inner diameter as described above.

Therefore, the disposition relationship between the rotary shaft 50 and the through-hole 74 will be described using FIG. 9 in conjunction. As shown in FIG. 9, the arc part 50C of the rotary shaft 50 is in sliding contact with the respective inner surfaces 52A and 74A of the through-hole 52 and the through-hole 74. The void portions 53 and 75 are provided between the side part 50B of the rotary shaft 50 and the respective inner surfaces 52A and 74A of the through-holes 52 and 74. The three protruding portions 84 of the sealing member 80 are fitted into the void portions 53 and 75. The through-hole 74 is an example of a second through-hole of the embodiment of the present invention.

Since the coupling structure of the present example comprises the sealing member 80, a rotation (swing) force of the lever 54 is first transmitted to the sealing member 80 and then transmitted from the sealing member 80 to the rotary shaft 50. Therefore, in the coupling structure of the present example, in order to transmit the rotation force of the lever 54 to the rotary shaft 50, for example, the lever 54 (through-hole 74) and the sealing member 80 (protruding portion 84) are fitted to each other with a dimensional relationship (interference fit) in which the lever 54 and the sealing member 80 are non-rotatable relative to each other. Consequently, it is possible to prevent the lever 54 and the sealing member 80 from freely rotating relative to each other, thereby enabling the rotation force of the lever 54 to be transmitted to the rotary shaft 50 via the sealing member 80. In addition, on a through-hole 52 side, in order to smoothly rotate the rotary shaft 50 (including the sealing member 80) with respect to the through-hole 52, the inner surface 52A of the through-hole 52 can be coated with a lubricating material, or a gap between the inner surface 52A and the sealing member 80 can also be filled with a lubricant.

Next, a coupling procedure between the elevator 26 and the lever 54 based on the coupling structure of the present example will be described. First, the sealing member 80 is mounted on the rotary shaft 50. Next, the sealing member 80 is mounted in the through-hole 74 of the lever 54. After that, the rotary shaft 50 is inserted through the through-hole 52 while the sealing member 80 is mounted in the through-hole 52 from a lever housing chamber 56 side, and the rotary shaft 50 that has penetrated the through-hole 52 is inserted into the through-hole 72 of the elevator 26. This procedure allows the elevator 26 and the lever 54 to be coupled to each other.

As alternative coupling procedures, the following two coupling procedures are exemplified. In a first alternative coupling procedure, first, the rotary shaft 50 is inserted through the through-hole 52 and is inserted into the through-hole 72. Next, the rotary shaft 50 is inserted into the through-hole 74. In this state, the sealing member 80 is mounted in the through-hole 74 and the through-hole 52 from a lever 54 side. This procedure also allows the elevator 26 and the lever 54 to be coupled to each other. In a second alternative coupling procedure, first, the elevator 26 is housed in the elevator housing chamber 28, the rotary shaft 50 is inserted through the through-hole 52 from the lever housing chamber 56 side and is inserted into the through-hole 72, and the rotary shaft 50 is attached and fixed to the elevator 26. After that, the lever 54 is attached to the rotary shaft 50 by inserting the rotary shaft 50 into the through-hole 74, and the lever 54 is housed in the lever housing chamber 56. Finally, the sealing member 80 is mounted in the through-hole 74 and the through-hole 52 from the lever 54 side in a state in which the lever 54, the rotary shaft 50, and the elevator 26 are assembled. This procedure also allows the elevator 26 and the lever 54 to be coupled to each other.

Next, a separation procedure between the elevator 26 and the lever 54 based on the coupling structure of the present example will be described. First, the lever 54 is removed from the lever housing chamber 56 side. Consequently, the sealing member 80 is removed from the lever housing chamber 56 side together with the lever 54. After that, the rotary shaft 50 is removed from the lever housing chamber 56 side. This procedure allows the elevator 26 and the lever 54 to be separated from each other. That is, the sealing member 80 of the present example is attachable and detachable from the lever housing chamber 56 side. Then, in a case in which separation work is completed, the inner surface 52A of the through-hole 52 is exposed, allowing for cleaning the inner surface 52A with the cleaning liquid.

Next, a coupling structure between the rotary shaft 50 and the elevator 26 will be described. As shown in FIG. 7, the elevator 26 is provided with the through-hole 72 into which the rotary shaft 50 is inserted. The rotary shaft 50 is formed with the side part 50B and the arc part 50C as described above.

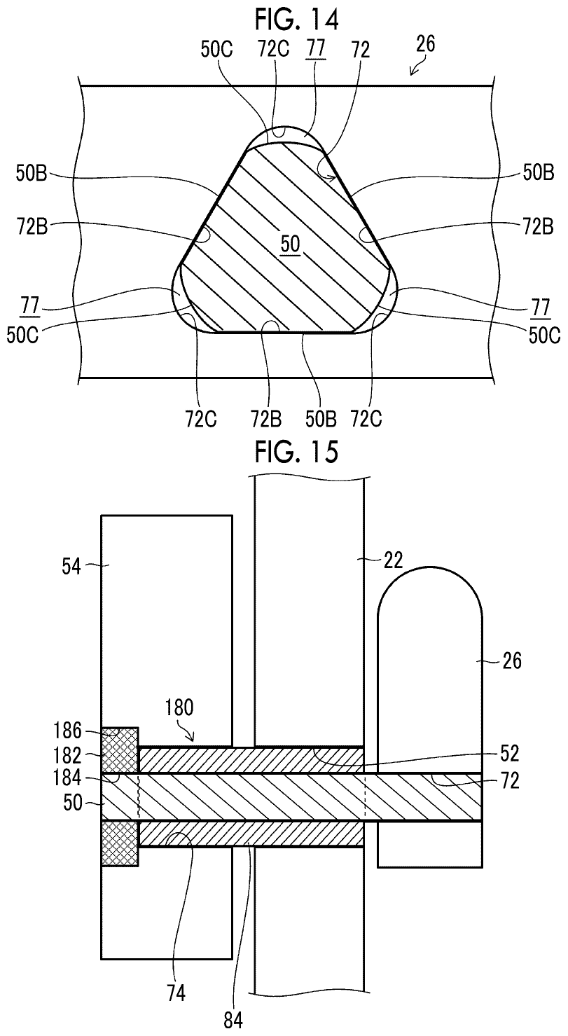

FIG. 14 is a cross-sectional view of the rotary shaft 50 and the through-hole 72.

As shown in FIG. 14, the through-hole 72 includes an inner peripheral portion formed with a hole-side side part 72B that faces and is in contact with the side part 50B, and a hole-side arc part 72C that faces the arc part 50C with a void portion 77 therebetween. The through-hole 72, the hole-side side part 72B, and the hole-side arc part 72C are examples of a third through-hole, a hole-side side part, and a hole-side arc part of the embodiment of the present invention.

With the above-described coupling structure, since the side part 50B of the rotary shaft 50 and the hole-side side part 72B of the through-hole 72 are in contact with each other, the rotation force transmitted from the lever 54 to the rotary shaft 50 can be transmitted to the elevator 26. In addition, since the void portion 77 formed between the arc part 50C and the hole-side arc part 72C can be used as a cleaning channel for allowing passage of the cleaning liquid, the cleanability of the arc part 50C and the hole-side arc part 72C exposed at the void portion 77 can be enhanced.

As described above, with the distal end portion 10 of the first embodiment, the sealing member 80 is mounted on the rotary shaft 50 that couples the elevator 26 and the lever 54 to each other, and the through-hole 52 is sealed by the sealing member 80 in a case in which the rotary shaft 50 is inserted through the through-hole 52, so that it is possible to prevent the liquid such as the body fluid from entering the lever housing chamber 56 from the through-hole 52. In addition, since the sealing member 80 is attachable and detachable from the lever housing chamber 56 side, the inner surface 52A of the through-hole 52 can be cleaned with the cleaning liquid. As a result, it is possible to improve the cleanability of the distal end portion 10.

### MODIFICATION EXAMPLES

Next, some modification examples (a first modification example and a second modification example) of the distal end portion 10 of the first embodiment will be described. In describing the modification examples, the same members as or similar members to those in the first embodiment will be designated by the same reference numerals.

### First Modification Example

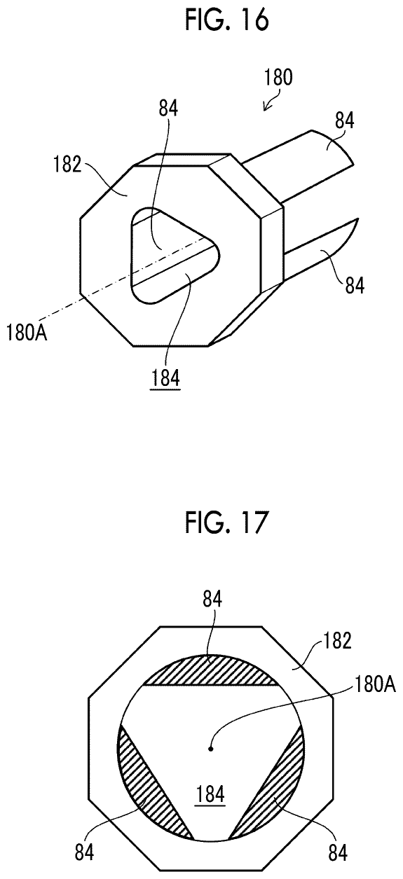

FIG. 15 is a cross-sectional view showing the first modification example in the coupling structure between the elevator 26 and the lever 54. FIG. 16 is a perspective view of a sealing member 180 applied to the coupling structure of FIG. 15.

As shown in FIGS. 15 and 16, the sealing member 180 comprises a base portion 182 and the three protruding portions 84 protruding from the base portion 182. The three protruding portions 84 are respectively fitted into the three void portions 53 shown in FIG. 10 to seal the through-hole 52. This point is the same as the sealing member 80 of the first embodiment.

A difference in the configuration between the sealing member 180 and the sealing member 80 is that the base portion 82 of the sealing member 80 has a disk shape, whereas the base portion 182 of the sealing member 180 is configured in a polygonal shape (an octagonal shape in the present example) in a front view as seen from a direction of an axis 180A shown in FIG. 17, and that a substantially triangular fitting hole 184 to which the rotary shaft 50 is fittable is provided. In FIG. 17, the protruding portion 84 is shown in a cross-section in order to distinguish between the base portion 182 and the protruding portion 84.

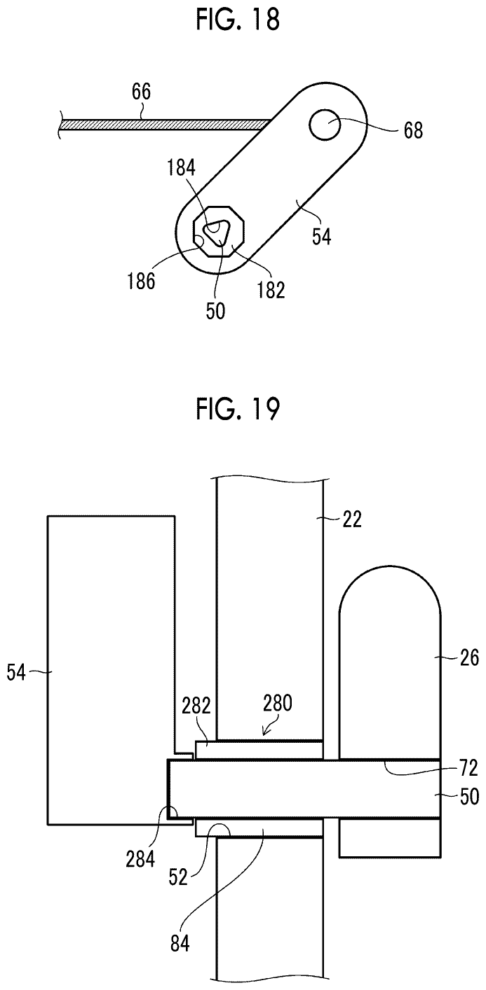

The base portion 182 is fitted to a fitting recess 186 formed in the lever 54, as shown in a side view of the lever 54 shown in FIG. 18. The fitting recess 186 is formed in a surface of the lever 54 on a side opposite to a facing surface side facing the first partition wall 22 and communicates with the through-hole 74.

The fitting recess 186 is configured in a polygonal shape (octagonal shape), similarly to the base portion 182. By fitting the base portion 182 to the fitting recess 186, the sealing member 180 is connected to the lever 54 to be non-rotatable relative to each other. That is, the sealing member 180 comprises the base portion 182 that is fitted to the lever 54 to be non-rotatable relative to each other. The base portion 182 is an example of a base portion of the embodiment of the present invention.

In addition, an end part of the rotary shaft 50 on the lever 54 side is fitted to the fitting hole 184 of the base portion 182. That is, the base portion 182 is provided with the fitting hole 184 to which the rotary shaft 50 is fitted to be non-rotatable relative to each other. The fitting hole 184 is an example of a fitting hole of the embodiment of the present invention.

According to the first modification example comprising such a sealing member 180, the base portion 182 of the sealing member 180 is fitted to the fitting recess 186 of the lever 54, and the rotary shaft 50 is fitted to the fitting hole 184 of the base portion 182, so that the rotation force of the lever 54 can be reliably transmitted to the sealing member 180 and the rotary shaft 50. In addition, the sealing member 180 is attachable and detachable from the lever housing chamber 56 side, similarly to the sealing member 80.

For the sealing member 180, in a case in which the base portion 182 is made of metal and the protruding portion 84 is made of a resin or rubber, the sealing member 180 can be manufactured by using a known insert molding method. By making the base portion 182 of metal, it is possible to enhance strength of the base portion 182, and by making the protruding portion 84 of a resin or rubber, it is possible to enhance adhesiveness of the protruding portion 84 to the through-holes 52 and 74.

### Second Modification Example

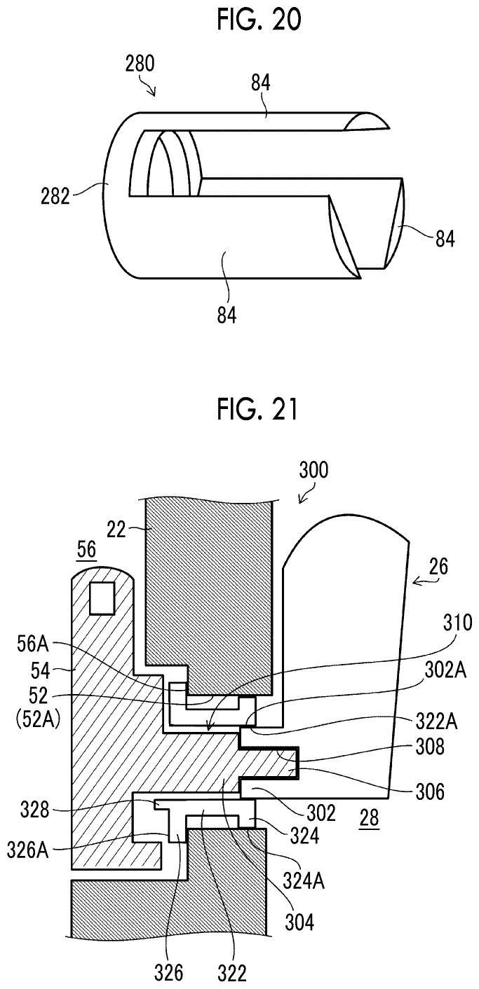

FIG. 19 is a cross-sectional view showing the second modification example in the coupling structure between the elevator 26 and the lever 54. FIG. 20 is a perspective view of a sealing member 280 applied to the coupling structure of FIG. 19.

As shown in FIGS. 19 and 20, the sealing member 280 comprises a base portion 282 and the three protruding portions 84 protruding from the base portion 282. The three protruding portions 84 are respectively fitted into the three void portions 53 shown in FIG. 10 to seal the through-hole 52. This point is the same as the sealing member 80 of the first embodiment.

A difference in the configuration between the first embodiment and the second modification example is that, as shown in FIG. 19, the lever 54 does not include the through-hole 74 (refer to FIG. 8) and includes a fitting recess 284 fitted to the rotary shaft 50 instead of the through-hole 74, and the base portion 282 of the sealing member 280 is disposed in a gap between the first partition wall 22 and the lever 54, while the base portion 82 of the sealing member 80 is disposed to penetrate the through-hole 74.

The fitting recess 284 is configured in a substantially triangular shape corresponding to the cross-sectional shape of the rotary shaft 50. The end part of the rotary shaft 50 on the lever 54 side is fitted to the fitting recess 284. That is, the lever 54 comprises the fitting recess 284 to which the rotary shaft 50 is fitted to be non-rotatable relative to each other.

According to the second modification example comprising such a lever 54, the rotary shaft 50 is fitted to the fitting recess 284, so that the rotation force of the lever 54 can be reliably transmitted to the rotary shaft 50. In addition, the sealing member 280 can be mounted from the lever housing chamber 56 side together with the lever 54. Further, the sealing member 280 can be removed from the lever housing chamber 56 side by removing the lever 54 from the lever housing chamber 56 side. In this case, the sealing member 280 is removed from the through-hole 52 by pinching the base portion 282 exposed in the lever housing chamber 56. That is, the sealing member 280 is attachable and detachable from the lever housing chamber 56 side. It should be noted that, in the first embodiment (including the first and second modification examples), the sealing members 80, 180, and 280 each including the three protruding portions 84 have been described to correspond to the shape of the rotary shaft 50 having a substantially triangular prism shape, but the present invention is not limited to this.

For example, in a case in which the rotary shaft 50 has a semi-circular column shape, the protruding portion 84 may be configured in a semi-circular column shape. The protruding portion 84 is formed with one side part and one arc part. In addition, in a case in which the rotary shaft 50 has a circular column shape, the protruding portion 84 may be configured in a tubular shape. In either case, the above-described (semi-circular column-shaped or circular column-shaped) protruding portion 84 can be fitted into the void portion formed between the outer peripheral surface of the rotary shaft 50 and the inner surface 52A of the through-hole 52. Consequently, it is possible to seal the through-hole 52. In the through-hole 72 on an elevator 26 side, a shape (a semi-circular shape and a round hole) and a size that can be fitted with a dimensional relationship (interference fit) in which the through-hole 72 and the above-described (semi-circular column-shaped or circular column-shaped) rotary shaft 50 are non-rotatable relative to each other need only be employed.

### Second Embodiment

Next, a second embodiment will be described. In describing the second embodiment, the same members as or similar members to those in the first embodiment will be designated by the same reference numerals.

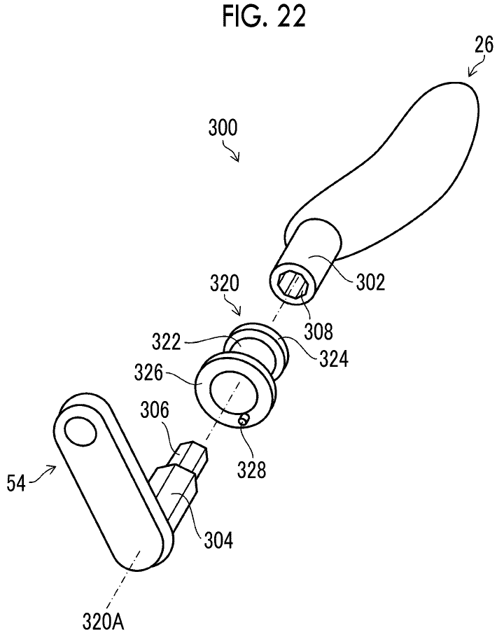

FIG. 21 is a cross-sectional view showing a main part structure of a distal end portion 300 according to the second embodiment, and, particularly, a coupling structure between the elevator 26 and the lever 54 is shown as a cross-sectional view. In addition, FIG. 22 is a perspective view of an assembly of the coupling structure shown in FIG. 21.

The first embodiment and the second embodiment are common to each other in that the rotary shaft that couples the elevator 26 and the lever 54 to each other is provided and the through-hole 52 through which the rotary shaft is inserted is sealed by the sealing member, but are different from each other in the configurations of the rotary shaft and the sealing member. Hereinafter, the difference will be mainly described.

First, the rotary shaft will be described. The rotary shaft 50 of the first embodiment employs the coupling structure in which the elevator 26 and the lever 54 are coupled to each other by a single rotary shaft 50. On the other hand, in the second embodiment, a single rotary shaft is configured with two rotary shafts coupled to each other, and a coupling structure that couples the elevator 26 and the lever 54 by means of the single rotary shaft is employed.

A specific description will be provided. As shown in FIGS. 21 and 22, in the second embodiment, an elevator-side rotary shaft 302 is provided on the elevator 26, and a lever-side rotary shaft 304 is provided on the lever 54. In the present example, the elevator 26 and the elevator-side rotary shaft 302 are an integrally formed article, and the lever 54 and the lever-side rotary shaft 304 are also an integrally formed article. The elevator-side rotary shaft 302 and the lever-side rotary shaft 304 are examples of a first rotary shaft and a second rotary shaft of the embodiment of the present invention.

A prismatic portion 306 having a smaller diameter than a diameter of the lever-side rotary shaft 304 is projected from the lever-side rotary shaft 304. The prismatic portion 306 is an octagonal prism body and is provided coaxially with the lever-side rotary shaft 304. That is, the lever 54 in the second embodiment is provided with the lever-side rotary shaft 304 including the prismatic portion 306. The prismatic portion 306 is an example of a fitting portion of the embodiment of the present invention.

Meanwhile, a prismatic recess portion 308 fitted to the prismatic portion 306 is provided on the elevator-side rotary shaft 302. The prismatic recess portion 308 is an octagonal bottomed hole and is provided coaxially with the elevator-side rotary shaft 302. That is, the elevator 26 in the second embodiment is provided with the elevator-side rotary shaft 302 including the prismatic recess portion 308. The prismatic recess portion 308 is an example of a fitted portion of the embodiment of the present invention.

With the coupling structure of the second embodiment configured as described above, in a case in which the elevator-side rotary shaft 302 and the lever-side rotary shaft 304 are inserted into the through-hole 52 from both sides of the through-hole 52 with the first partition wall 22 interposed therebetween, the prismatic portion 306 and the prismatic recess portion 308 are fitted to each other. As a result, the elevator 26 and the lever 54 are coupled to each other by a single rotary shaft 310 consisting of the elevator-side rotary shaft 302 and the lever-side rotary shaft 304, and the rotation force of the lever 54 can be transmitted to the elevator 26 via the rotary shaft 310.

Next, the sealing member will be described. The sealing member 80 of the first embodiment employs a sealing structure in which the protruding portion 84 provided on the base portion 82 is fitted into the void portion 53 formed in the through-hole 52 to seal the through-hole 52.

Meanwhile, a sealing member 320 of the second embodiment comprises a cylindrical portion 322 inserted through the through-hole 52, and flange portions 324 and 326 provided on both sides of the cylindrical portion 322. The flange portion 324 is positioned in the through-hole 52, and the flange portion 326 is positioned in the lever housing chamber 56, thereby employing the sealing structure that seals the through-hole 52.

A specific description will be provided. The sealing member 320 includes the cylindrical portion 322 having a tubular shape through which the elevator-side rotary shaft 302 and the lever-side rotary shaft 304 are inserted. An outer surface 302A of the elevator-side rotary shaft 302 is in sliding contact with an inner surface 322A of the cylindrical portion 322.

In addition, the sealing member 320 includes the flange portion 324. The flange portion 324 is provided on an elevator-side rotary shaft 302 side and protrudes in a radial direction orthogonal to a direction of an axis 320A. Additionally, the sealing member 320 includes the flange portion 326. The flange portion 326 is provided on a lever-side rotary shaft 304 side and protrudes in the above-described radial direction. Further, the flange portion 326 is configured to have a larger diameter than a diameter of the flange portion 324.

The flange portion 324 is located in the through-hole 52, and an outer peripheral surface 324A of the flange portion 324 is in contact with the inner surface 52A of the through-hole 52. In addition, the flange portion 326 is located in the lever housing chamber 56 and is in contact with a bottom portion 56A of the lever housing chamber 56. Further, a knob portion 328 is provided to protrude at an end part of the sealing member 320 on the lever housing chamber 56 side, specifically, at an end part 326A of the flange portion 326 on the lever housing chamber 56 side. The knob portion 328 protrudes toward the lever housing chamber 56 side. The flange portion 324, the flange portion 326, and the knob portion 328 are examples of a first flange portion, a second flange portion, and a knob portion of the embodiment of the present invention.

### Mounting Method of Sealing Member in Through-Hole

The sealing member 320 is pushed into the through-hole 52 from the lever housing chamber 56 side with the flange portion 324 as a leading end. Consequently, the sealing member 320 can be mounted in the through-hole 52. In this case, the flange portion 324 is located in the through-hole 52, and the flange portion 326 is located in the lever housing chamber 56. After that, as described above, in a case in which the elevator 26 and the lever 54 are coupled to each other by the rotary shaft 310, the outer surface 302A of the elevator-side rotary shaft 302 is in sliding contact with the inner surface 322A of the cylindrical portion 322. As a result, it is possible to seal the through-hole 52.

Removal Method of Sealing Member from Through-Hole

First, the lever 54 is removed from the lever housing chamber 56 side. After that, the sealing member 320 is removed from the through-hole 52 by pinching the knob portion 328. As a result, it is possible to clean the inner surface 52A of the through-hole 52.

Therefore, in the distal end portion 300 of the second embodiment as well, similarly to the distal end portion 10 of the first embodiment, the through-hole 52 is sealed by the sealing member 320, so that it is possible to prevent the liquid such as the body fluid from entering the lever housing chamber 56 from the through-hole 52. In addition, since the sealing member 320 is attachable and detachable from the lever housing chamber 56 side, it is possible to clean the inner surface 52A of the through-hole 52 with the cleaning liquid. As a result, it is possible to improve the cleanability of the distal end portion 300.

### Attachment and Detachment Form of Sealing Member

It is preferable that the sealing member 320 of the second embodiment is attachable to and detachable from at least the lever-side rotary shaft 304. Consequently, the lever-side rotary shaft 304 and the sealing member 320 can be inserted through the through-hole 52 together in a state in which the sealing member 320 is mounted on the lever-side rotary shaft 304 in advance. The sealing member 320 may be attachably and detachably provided on the elevator-side rotary shaft 302.

### Fixing Form of Sealing Member

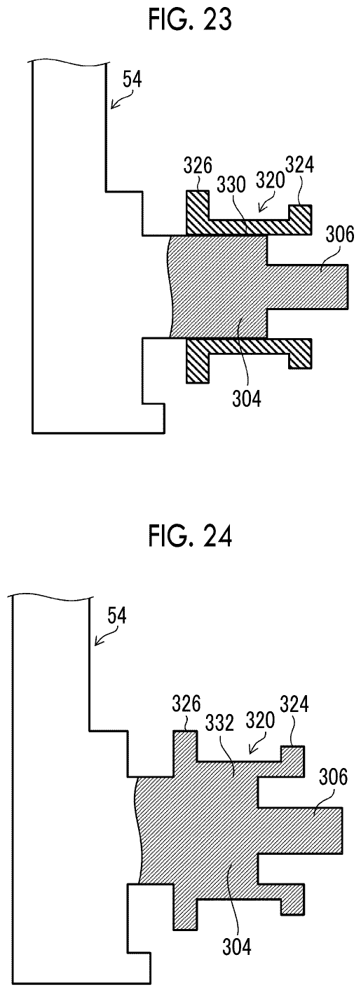

The sealing member 320 may be fixed to the lever-side rotary shaft 304. FIG. 23 is a cross-sectional view of the sealing member 320 fixed to the lever-side rotary shaft 304 with an adhesive 330. In this case as well, the lever-side rotary shaft 304 and the sealing member 320 can be inserted through the through-hole 52 together. In addition, the lever-side rotary shaft 304 and the sealing member 320 can be removed together from the through-hole 52.

### Form of Integrally Formed Article of Sealing Member

In addition, the sealing member 320 and the lever-side rotary shaft 304 may be an integrally formed article. FIG. 24 is a cross-sectional view of the sealing member 320 and the lever-side rotary shaft 304 that are configured as an integrally formed article 332. In this case as well, it is possible to obtain the same effect as in the above-described fixing form.

It should be noted that, in the second embodiment, the prismatic recess portion 308, which is the fitted portion, is provided on the elevator-side rotary shaft 302, and the prismatic portion 306, which is the fitting portion, is provided on the lever-side rotary shaft 304, but the present invention is not limited to this, and the prismatic portion 306 may be provided on the elevator-side rotary shaft 302, and the prismatic recess portion 308 may be provided on the lever-side rotary shaft 304.

In addition, in the second embodiment, a fitting form between the prismatic portion 306 and the prismatic recess portion 308 has been described as a fitting form between the fitting portion and the fitted portion, but the present invention is not limited to this, and, for example, a fitting form between a circular column portion and a round recess portion may be used as long as it is a fitting form capable of reliably transmitting the rotation force of the lever 54 to the elevator 26.

Further, in the second embodiment, a form has been described in which the elevator 26 and the elevator-side rotary shaft 302 are an integrally formed article, but the present invention is not limited to this. For example, the elevator 26 and the elevator-side rotary shaft 302 may be separately configured, and the elevator 26 and the elevator-side rotary shaft 302 may be coupled to each other by the coupling structure described above with reference to FIG. 14. That is, a coupling structure may be employed in which the through-hole 72 is provided in the elevator 26 and the side part 50B and the arc part 50C are formed on the elevator-side rotary shaft 302.

In each of the above-described embodiments, the side-viewing endoscope has been described as an example, but the present invention can be applied to various endoscopes such as an ultrasonic endoscope and a direct-viewing endoscope, which comprise an elevator that is provided at the distal end portion of the insertion part and that changes the lead-out direction of the treatment tool.

Although the endoscope according to the embodiment has been described above, the present invention may be subjected to some improvements or modifications without departing from the gist of the present invention.

### EXPLANATION OF REFERENCES

10: distal end portion
12: insertion part
14: bending portion
16: cylindrical member
20: distal end portion body
22: first partition wall
24: second partition wall
24A: distal end surface
26: elevator
28: elevator housing chamber
30: opening
32: treatment tool insertion channel
32A: treatment tool outlet port
50: rotary shaft
50A: axis
50B: side part
50C: arc part
52: through-hole
52A: inner surface
53: void portion
53A: void-side side part
53B: void-side arc part
54: lever
56: lever housing chamber
58: housing chamber
60: irradiation window
62: observation window
64: air and water supply nozzle
66: operation wire
68: connection portion
70: outlet
71: wire insertion passage
72: through-hole
72B: hole-side side part
72C: hole-side arc part
74: through-hole
74A: inner surface
75: void portion
77: void portion
80: sealing member
80A: axis
82: base portion
84: protruding portion
84A: protruding-side side part
84B: protruding-side arc part
86: slit
88: shaft housing space
100: distal end cover
102: open part
180: sealing member
182: base portion
184: fitting hole
186: fitting recess
200: cap
202: opening window
280: sealing member
282: base portion
284: fitting recess
300: distal end portion
302: elevator-side rotary shaft
302A: outer surface
304: lever-side rotary shaft
306: prismatic portion
308: prismatic recess portion
310: rotary shaft
320: sealing member
320A: axis
322: cylindrical portion
322A: inner surface
324: flange portion
326: flange portion
330: adhesive
332: integrally formed article
Ax: major axis
G: guide surface

What is claimed is:

1. An endoscope comprising:
a distal end portion including an elevator housing part and a lever housing part;
an elevator housed in the elevator housing part;
a lever housed in the lever housing part;
a wire connected to the lever;
a partition wall that is provided at the distal end portion and that divides the elevator housing part and the lever housing part;
a rotary shaft that couples the elevator and the lever to each other, the rotary shaft being inserted through a first through-hole provided in the partition wall; and
a sealing member that is mounted on the rotary shaft and that seals the first through-hole in a case in which the rotary shaft is inserted through the first through-hole, the sealing member being attachable and detachable from a lever housing part side.

2. The endoscope according to claim 1,
wherein the lever includes a second through-hole through which the rotary shaft is inserted,
the rotary shaft is formed with a side part in which a peripheral surface parallel to an axial direction of the rotary shaft forms a planar shape, and an arc part in which the peripheral surface forms a curved shape,
the arc part is in sliding contact with an inner surface of each of the first through-hole and the second through-hole,
a void portion is provided between the side part and the inner surface of each of the first through-hole and the second through-hole, and
the sealing member is fitted into the void portion.

3. The endoscope according to claim 2,
wherein the sealing member includes
a base portion that is fitted to the lever to be non-rotatable relative to each other, and
a protruding portion protruding from the base portion, and
the protruding portion is fitted into the void portion.

4. The endoscope according to claim 3,
wherein the base portion includes a fitting hole to which the rotary shaft is fitted to be non-rotatable relative to each other.

5. The endoscope according to claim 1, further comprising:
a first rotary shaft that is provided on the elevator and that constitutes the rotary shaft; and
a second rotary shaft that is provided on the lever and that constitutes the rotary shaft,
wherein a fitting portion is provided on one of the first rotary shaft or the second rotary shaft, and a fitted portion that is fitted to the fitting portion is provided on the other rotary shaft different from the one rotary shaft.

6. The endoscope according to claim 5,
wherein the sealing member is attachable to and detachable from at least the second rotary shaft.

7. The endoscope according to claim 5,
wherein a knob portion is provided to protrude at an end part of the sealing member on the lever housing part side.

8. The endoscope according to claim 5,
wherein the sealing member is fixed to the second rotary shaft.

9. The endoscope according to claim 5,
wherein the sealing member and the second rotary shaft are an integrally formed article.

10. The endoscope according to claim 5,
wherein the sealing member has a tubular shape through which the first rotary shaft and the second rotary shaft are inserted, and
the sealing member includes a first flange portion that is provided on a first rotary shaft side in an axial direction of the sealing member and that protrudes in a radial direction orthogonal to the axial direction, and a second flange portion that is provided on a second rotary shaft side in the axial direction and that protrudes in the radial direction.

11. The endoscope according to claim 10,
wherein a knob portion is provided to protrude at an end part of the second flange portion on the lever housing part side.

12. The endoscope according to claim 10,
wherein the first flange portion is disposed in the first through-hole, and
the second flange portion is disposed in the lever housing part.

13. The endoscope according to claim 1,
wherein the elevator includes a third through-hole into which the rotary shaft is inserted,
the rotary shaft is formed with a side part in which a peripheral surface parallel to an axial direction of the rotary shaft forms a planar shape, and an arc part in which the peripheral surface forms a curved shape, and
the third through-hole includes an inner peripheral portion formed with a hole-side side part that faces and is in contact with the side part and a hole-side arc part that faces the arc part with a void portion therebetween.
